SEQUENCE LISTING (8 sequences)

SEQ ID NO: 1 - DNA, 1423 bp, synthetic construct
  source 1..1423: mol_type = other DNA
tggaggctactcttaccatgcaagtcgacggaaaggccccttcgggggtgctcgagtggcgaacgggtgagtaacacgtgggtgatctgccctgcactctgggataagcctgggaaactgggtctaataccggatacacccttctggttgcatggctgggaggggaaagcttttgcggtgtgggatgggcccgcggcctatcagcttgttggtggggtgatggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgggggatgacggccttcgggttgtaaacccctttcagtagggacgaagcgtgagtgacggtacctacagaagaagcaccggccaactacgtgccagcagccgcggtaatacgtagggtgcgagcgttgtccggaattactgggcgtaaagagctcgtaggtggtttgtcgcgttgttcgtgaaaactcacagcttaactgtgggcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgacggcagagatgtcgtttcccttgtggcctgtgtgcaggtggtgcatggctgtcgtcagctcgtgtcgtgagatgttgggttaagtcccgcaacgagcgcaacccttgtctcatgttgccagcacgttatggtggggactcgtgagagactgccggggtcaactcggaggaaggtggggatgacgtcaagtcatcatgccccttatgtccagggcttcacacatgctacaatggccggtacaaagggctgcgatgccgtgaggtggagcgaatcctttcaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggaggcagccgtcgaaggaataccccc

SEQ ID NO: 2 - DNA, 22 bp, synthetic construct
  source 1..22: mol_type = other DNA
gtaggtggtttgtcgcgttgtt

SEQ ID NO: 3 - DNA, 22 bp, synthetic construct
  source 1..22: mol_type = other DNA
cacctagtacccaccgtttacg

SEQ ID NO: 4 - DNA, 25 bp, synthetic construct
  source 1..25: mol_type = other DNA
tacttaatgcgttagctacggcacg

SEQ ID NO: 5 - DNA, 23 bp, synthetic construct
  source 1..23: mol_type = other DNA
ggcggggtacttaatgcgttagc

SEQ ID NO: 6 - DNA, 18 bp, synthetic construct
  source 1..18: mol_type = other DNA
gccatgcaccacctgcac

SEQ ID NO: 7 - DNA, 19 bp, synthetic construct
  source 1..19: mol_type = other DNA
ccctttgtaccggccattg

SEQ ID NO: 8 - DNA, 16 bp, synthetic construct
  source 1..16: mol_type = other DNA
attcctggtgtagcgg

(19) United States
(12) Patent Application Publication
(10) Pub. No.: US 2025/0215511 A1
(43) Pub. Date: Jul. 3, 2025

(54) MYCOBACTERIUM DNA DETECTION PRIMER AND DETECTION METHOD

(71) Applicant: HUZHOU SHENKE BIOTECHNOLOGY CO., LTD., Huzhou, Zhejiang (CN)

(72) Inventors: Wanxin WU, Huzhou, Zhejiang (CN); Danni CHENG, Huzhou, Zhejiang (CN); Fen MENG, Huzhou, Zhejiang (CN); Weiying ZONG, Huzhou, Zhejiang (CN); Bin SHEN, Huzhou, Zhejiang (CN); Jia TANG, Huzhou, Zhejiang (CN)

(21) Appl. No.: 18/850,330

(22) PCT Filed: Mar. 27, 2023
(86) PCT No.: PCT/CN2023/084179

§ 371 Date: Sep. 24, 2024

(30) Foreign Application Priority Data
Mar. 25, 2022 (CN) 202210306869.3

(51) Int. Cl. C12Q 1/689; C07H 21/00

(52) U.S. Cl. CPC C12Q 1/689; C07H 21/00

(57) ABSTRACT

The present invention provides a primer pair for detecting a Mycobacterium genomic DNA, a detection reagent containing the primer pair of the present invention, and a method for detecting the Mycobacterium genomic DNA by using the primer pair, wherein the primer pair specifically binds to a sequence as shown in SEQ ID NO: 1.

### TECHNICAL FIELD

The present invention relates to the field of bioassays. In particular, the present invention relates to primers and methods for detecting Mycobacterium DNA.

### BACKGROUND

With the growing international concern on the risk of Mycobacterium contamination, newly revised WHO and FDA guidelines for vaccine protocols in recent years have introduced requirements for testing Mycobacterium. In the WHO guideline, it is stipulated that biological raw materials used in the production of biologics, such as cellular matrices, should be tested to confirm that they are free of exogenous infectious pathogens, such as bacteria, fungi, culturable and non-culturable mycoplasmas, spirochetes (in the case of insect cells or cells exposed to plant-derived materials) and mycobacteria. To ensure the feasibility of the assay, the sensitivity of the assay must be validated in advance to confirm that the assay can accurately detect the presence of contaminants in the cellular matrix at detectable concentrations. If a cell is susceptible to be infected by Mycobacterium tuberculosis or other mycobacteria, the quality control of its cell bank should include a testing for mycobacteria, which is also required for primary cells. Given that some mycobacteria grow predominantly intracellularly, it is necessary to detect mycobacteria by lysing the host cells.

More than 200 Mycobacterium species have been reported. For safety reasons, the scope of mycobacterial testing for biological products must cover as many mycobacteria as possible, and those to be tested include viral vaccine seed lots, genetically engineered vaccine seed lots, as well as recombinant proteins and cell banks of monoclonal antibody products.

For live attenuated human encephalitis B vaccine, WHO requires that: the animal stocks, seed lots and single virus harvests for the production should be tested for Mycobacterium avium, either by traditional culture, guinea pig or by nucleic acid amplification (NAT), which must be validated and approved by the national regulatory authority prior to use. For live attenuated dengue vaccines, WHO requires that: seed lots of virulent seed and single virus harvests should be tested for mycobacteria, and the assay used should be validated by the national regulatory authority. NAT can be used as a modified method of mycobacterial culture method, but it must be approved by the national regulatory authority prior to use. For live attenuated human influenza vaccine nasally administered, WHO recommends that: each single harvest should be tested for Mycobacterium, commonly by centrifuging and concentrating the virus harvest and inoculating guinea pigs or solid media.

The FDA guideline requires that: Mycobacterium can be tested by a culture method, which can be performed on Roger's medium or other suitable media, and that the test should be accompanied with a positive control. The guinea pig method can also be used to detect Mycobacterium. If an improved method such as PCR is used, the used method should be sufficiently sensitive.

The Chinese Pharmacopoeia requires that all cell lines/strains used for the production of biological products must be subjected to a comprehensive check, must have the appropriate information, and must be approved by the State Drug Administration. Cell lysates were prepared from the culture supernatant of at least 10⁷ live cells and examined for mycobacteria according to the sterility testing method of the Chinese Pharmacopoeia. The cell lysates were inoculated in a suitable solid medium (e.g. Roche medium (LowensteinJensen medium) or Middlebrook 7H10 medium), 1 ml was inoculated for each medium in triplicate. And at the same time, Mycobacterium grassii was used as a positive control. The inoculated medium was incubated at 37° C. for 56 days, the positive control should exhibit bacterial growth, and no growth of Mycobacterium avium was found in the medium inoculated with the test article, then it was judged to be qualified.

The guinea pig inoculation method used for exogenous virus detection can also detect mycobacteria, therefore the guinea pig method can also be used for mycobacteria detection. The guinea pigs should be observed for 4 weeks before injection, and only those with negative tuberculin test can be used for the test. At the end of the observation period, tuberculin test should be carried out, and autopsy should be performed to observe whether there are nodules formed in the main organs. If the tuberculin test is negative and there are no nodules in the main organs, then the requirements are met. Mycobacteria testing can also be performed using a validated mycobacteria nucleic acid test instead of culture.

With the development of technology and new forms of biologics, such as cell therapy and gene therapy products widely available, rapid test methods for Mycobacterium, especially the NAT method, which has advantages in sensitivity, specificity and timeliness, are increasingly being paid attention to and used by the industry. It is pointed out in the WHO and FDA guidelines and the Chinese Pharmacopoeia that, if it is proved that the NAT method for Mycobacterium is applicable through suitable method validation, then the NAT method can be used to replace the culture method or guinea pig method.

In summary, there is an urgent need in the art for methods to detect Mycobacterium DNA, which should have advantages of specificity, sensitivity, simplicity of operation, and standardization.

### SUMMARY OF THE INVENTION

The purpose of the present invention is to provide a primer pair for detecting Mycobacterium DNA with high sensitivity and high specificity, as well as a detection reagent or PCR kit comprising said primer pair.

Another purpose of the present invention is to provide methods or PCR methods for detecting Mycobacterium genomic DNA using the primer pair or detection reagent provided herein.

In a first aspect, a primer pair is provided in the present invention for detecting Mycobacterium genomic DNA, said primer pair comprising a forward primer and a reverse primer, wherein the forward primer binds to positions 510-550, preferably positions 518-539, of the sequence shown in SEQ ID NO: 1 on the Mycobacterium genomic DNA; the reverse primer binds to positions 740-780, 780-830, 790-830, 970-1010 or 1160-1200, preferably positions 748-769, 791-815, 800-822, 979-996, or 1175-1193, of the sequence shown in SEQ ID NO: 1 on the Mycobacterium genomic DNA; and the length of the product obtained through the amplification by said primer pairs is 240 to 700 bp, respectively.

In a preferred embodiment, the forward and reverse primers are in a length of 18-25 bp; preferably 20 bp.

In a preferred embodiment, the forward and reverse primers have a Tm temperature of 61-63° C. and the absolute value of the difference between the Tm of the forward primer and the Tm of the reverse primer is ≤1° C.

In a specific embodiment, in the primer pair, the forward primer is shown in SEQ ID NO: 2 and the reverse primer is shown in any one of SEQ ID NO: 3-7.

In a specific embodiment, in the primer pair, the forward primer is shown in SEQ ID NO: 2 and the reverse primer is shown in SEQ ID NO: 6.

In a specific embodiment, the primer pair further comprises a probe, wherein the probe binds to positions 600-640, preferably positions 613-628 of the sequence shown in SEQ ID NO: 1 on the genomic DNA of Mycobacterium.

In a specific embodiment, the probe is shown in SEQ ID NO: 8.

In a second aspect, a detection reagent is provided in the present invention, the detection reagent comprising the primer pair described in the first aspect.

In a preferred embodiment, the detection reagent has a detection sensitivity of 10 CFU of Mycobacterium.

In a third aspect, a method for detecting genomic DNA of Mycobacterium is provided in the present invention, said method comprising: subjecting a sample to be tested to a PCR using the primer pair as described in the first aspect or detection reagent as described in the second aspect and detecting a PCR amplification product.

In a fourth aspect, a PCR kit is provided in the present invention, the kit comprising a container and the primer pair described in the first aspect in the container.

In a preferred embodiment, the forward and reverse primers are in a length of 18-25 bp; preferably 20 bp.

In a preferred embodiment, the forward and reverse primers have a Tm temperature of 61-63° C. and the absolute value of the difference between the Tm of the forward primer and the Tm of the reverse primer is ≤1° C.

In a preferred embodiment, in the primer pair, the forward primer is as shown in SEQ ID NO: 2 and the reverse primer is shown in any one of SEQ ID NO: 3-7.

In a preferred embodiment, the kit further comprises a probe

In a preferred embodiment, the probe is shown in SEQ ID NO: 8.

In a preferred embodiment, the detection reagent has a detection sensitivity of 10 CFU of Mycobacterium.

In a preferred embodiment, the kit further comprises other reagents required to perform a PCR amplification and optionally a standard control.

In a fifth aspect, a PCR method is provided in the present invention, comprising a step of:
amplifying a target product in a PCR detection system utilizing a primer pair as described in the first aspect.

In a preferred embodiment, the forward and reverse primers are in a length of 18-25 bp; preferably 20 bp.

In a preferred embodiment, the forward and reverse primers have a Tm temperature of 61-63° C. and the absolute value of the difference between the Tm of the forward primer and the Tm of the reverse primer is ≤1° C.

In a preferred embodiment, in the primer pair, the forward primer is as shown in SEQ ID NO: 2 and the reverse primer is shown in any one of SEQ ID NO: 3-7.

In a preferred embodiment, the PCR detection further comprises a probe

In a preferred embodiment, the probe is shown in SEQ ID NO: 8.

In a sixth aspect, a use of a primer pair as described in the first aspect or an detection reagent as described in the second aspect is provided in the present invention for detecting the presence of Mycobacterium DNA in a subject to be tested.

In a preferred embodiment, the subject to be tested is a vaccine, preferably a live attenuated human encephalitis B vaccine.

It should be understood that, within the scope of the present invention, each of the above-described technical features of the present invention and each of the technical features specifically described below (e.g., in the Examples) can be combined with each other, thereby constituting a new or preferred technical solution, which shall not be repeated herein one by one due to the limited contents.

### DESCRIPTION OF DRAWINGS

FIG. 1 shows the amplification curve of the reference, wherein the used primer pair is SEQ ID NO: 2, SEQ ID NO: 6.

FIG. 2 shows the results of an interference experiment, wherein the used primer pair is SEQ ID NO: 2, SEQ ID NO: 6.

### MODES FOR CARRYING OUT THE INVENTION

After an in-depth and extensive research, the present inventors unexpectedly found that the primers designed for the region shown in SEQ ID NO: 1 of the genomic DNA of Mycobacterium can not only detect the genomic DNA of Mycobacterium with high sensitivity, but also differentiate interfering DNAs from a variety of common bacteria, fungi, Mycoplasma, engineered cells, and bacteria that have a close evolution relationship with Mycobacterium, etc. The method of the present invention is simple, rapid, highly specific, and sensitive, based on which the present invention has been accomplished.

### Primer Pair of the Present Invention

The term “primer” as used herein has the meaning conventionally understood by a skilled person. The primers specific for Mycobacterium genomic DNA of the present invention are not designed for the exogenous gene itself or for the viral vector itself, but are designed for the region shown in SEQ ID NO: 1 on the Mycobacterium genomic DNA. In other words, the primers of the present invention can specifically bind to the region shown in SEQ ID NO: 1 on the Mycobacterium genomic DNA.

In view of the teachings of the present invention and common knowledge in the art, it should be understood by a skilled person that a variety of primer pairs can be designed for the region shown in SEQ ID NO: 1. Therefore, the primer pairs of the present invention are not limited to the primer pairs specifically obtained in the Examples.

In a specific embodiment, the forward primer of the present invention binds to positions 510-550, preferably positions 518-539, of the sequence shown in SEQ ID NO: 1; the reverse primer binds to positions 740-780, 780-830, 790-830, 970-1010 or 1160-1200, preferably positions 748-769, 791-815, 800-822, 979-996, or 1175-1193, of the sequence shown in SEQ ID NO: 1; and the length of the product obtained through the amplification by said primer pairs is 240 to 700 bp, respectively.

In a preferred embodiment, the forward and reverse primers are in a length of 18-25 bp; preferably 20 bp.

In a preferred embodiment, the forward and reverse primers have a Tm temperature of 61-63° C. and the absolute value of the difference between the Tm of the forward primer and the Tm of the reverse primer is ≤1° C.

In a specific embodiment, in the primer pair, the forward primer is shown in SEQ ID NO: 2 and the reverse primer is shown in any one of SEQ ID NO: 3-7.

### Probe

The term “probe” as used herein has the meaning conventionally understood by a skilled person, that is, a small fragment of single-stranded DNA or RNA that is used to detect a nucleic acid sequence complementary to it.

In view of the teachings of the present invention and common knowledge in the art, a skilled person should understand that, if a primer pair is known, a skilled person can independently design a probe based on the template sequence between the forward primer and reverse primer binding site, and test the technical effect of the probe with the primer pair. In a specific embodiment, a skilled person can design a probe according to practical needs, and said probe can be in a liquid phase or fixed on a solid phase; it can bind to an amplified product before amplification or after amplification. Therefore, the probe of the present invention is not limited to the probe specifically disclosed in the Examples. And the primer pairs of the present invention are not limited to be used in pairing with the specifically disclosed probes of the Examples.

In a specific embodiment, the probe is shown in SEQ ID NO: 8.

### Detection Reagent of the Present Invention

A detection reagent is also provided in the present invention for detecting genomic DNAs of Mycobacterium. The detection reagent comprises the primer pairs of the present invention as well as probes and other components necessary for performing PCR, such as Taq enzyme, dNTP, Mg²⁺, and the like.

In a specific embodiment, the detection reagent of the present invention comprises the forward primer shown in SEQ ID NO: 2, the reverse primer shown in any of SEQ ID NO: 3-7, and the probe shown in SEQ ID NO: 8. In a preferred embodiment, the detection reagent of the present invention comprises the forward primer shown in SEQ ID NO: 2, the reverse primer shown in SEQ ID NO: 6.

In a specific embodiment, the detection reagents of the present invention have a detection sensitivity of 10 CFU of Mycobacterium.

Based on the primer pairs or detection reagents of the present invention, a method for detecting genomic DNA of Mycobacterium is further provided in the present invention, said method comprising: subjecting PCR on a sample to be tested by using the primer pairs or detection reagents of the present invention, and detecting the PCR amplification product.

Based on the primer pairs of the present invention, a PCR kit is also provided in the present invention, said kit comprising a container and the primer pairs of the present invention comprised in the container.

In a specific embodiment, other required components for performing PCR and instructions for using the kit for PCR detection is further comprised in the PCR kit of the present invention. In a preferred embodiment, a standard control is also comprised in the kit.

Based on the primer pairs of the present invention, a PCR method for amplifying a target product using the primer pairs of the present invention is provided in the present invention.

### Advantages of the Invention

1. the primer pairs or detection reagents of the present invention are capable of detecting Mycobacterium genomic DNAs with a high sensitivity;
2. the primer pairs or detection reagents of the present invention are capable of detecting Mycobacterium genomic DNAs with a high sensitivity;
3. the primer pairs or detection reagents of the present invention are capable of distinguishing interfering DNAs from Streptococcus pneumoniae, Streptococcus icterus, Candida albicans, Aspergillus niger, etc;
4. the detection method of the present invention is simple and rapid in operation, and exhibits high specificity and sensitivity.

The present invention will be further described below in connection with specific Examples. It should be understood that these Examples are used only to illustrate the invention and are not intended to limit the scope of the invention. Experimental methods for which specific conditions are not indicated in the following Examples are generally in accordance with conventional conditions, such as those described in Sambrook et al, Molecular Cloning: a Laboratory Manual (Cold Spring Harbor Laboratory Press, 2001), or in accordance with conditions recommended by a manufacturer. Percentages and servings are calculated by weight unless otherwise stated.

### EXAMPLE

### Example 1. Design of Primer Pair and Probe

The present inventors have designed primer pairs and probes for detecting Mycobacterium genomic DNA using following target sequences: >KF378762.1

Mycobacterium phlei strain CM32 16S
ribosomal RNA gene, partial sequence
(SEQ ID NO: 1)
TGGAGGCTACTCTTACCATGCAAGTCGACGGAAAGGCCCCTTCGGGGGTG
CTCGAGTGGCGAACGGGTGAGTAACACGTGGGTGATCTGCCCTGCACTCT
GGGATAAGCCTGGGAAACTGGGTCTAATACCGGATACACCCTTCTGGTTG
CATGGCTGGGAGGGGAAAGCTTTTGCGGTGTGGGATGGGCCCGCGGCCTA
TCAGCTTGTTGGTGGGGTGATGGCCTACCAAGGCGACGACGGGTAGCCGG
CCTGAGAGGGTGTCCGGCCACACTGGGACTGAGATACGGCCCAGACTCCT
ACGGGAGGCAGCAGTGGGGAATATTGCACAATGGGCGCAAGCCTGATGCA
GCGACGCCGCGTGGGGGATGACGGCCTTCGGGTTGTAAACCCCTTTCAGT
AGGGACGAAGCGTGAGTGACGGTACCTACAGAAGAAGCACCGGCCAACTA
CGTGCCAGCAGCCGCGGTAATACGTAGGGTGCGAGCGTTGTCCGGAATTA
CTGGGCGTAAAGAGCTCGTAGGTGGTTTGTCGCGTTGTTCGTGAAAACTC
ACAGCTTAACTGTGGGCGTGCGGGCGATACGGGCAGACTAGAGTACTGCA
GGGGAGACTGGAATTCCTGGTGTAGCGGTGGAATGCGCAGATATCAGGAG
GAACACCGGTGGCGAAGGCGGGTCTCTGGGCAGTAACTGACGCTGAGGAG
CGAAAGCGTGGGGAGCGAACAGGATTAGATACCCTGGTAGTCCACGCCGT
AAACGGTGGGTACTAGGTGTGGGTTTCCTTCCTTGGGATCCGTGCCGTAG
CTAACGCATTAAGTACCCCGCCTGGGGAGTACGGCCGCAAGGCTAAAACT
CAAAGGAATTGACGGGGGCCCGCACAAGCGGCGGAGCATGTGGATTAATT
CGATGCAACGCGAAGAACCTTACCTGGGTTTGACATGCACAGGACGACGG
CAGAGATGTCGTTTCCCTTGTGGCCTGTGTGCAGGTGGTGCATGGCTGTC
GTCAGCTCGTGTCGTGAGATGTTGGGTTAAGTCCCGCAACGAGCGCAACC
CTTGTCTCATGTTGCCAGCACGTTATGGTGGGGACTCGTGAGAGACTGCC
GGGGTCAACTCGGAGGAAGGTGGGGATGACGTCAAGTCATCATGCCCCTT
ATGTCCAGGGCTTCACACATGCTACAATGGCCGGTACAAAGGGCTGCGAT
GCCGTGAGGTGGAGCGAATCCTTTCAAAGCCGGTCTCAGTTCGGATCGGG
GTCTGCAACTCGACCCCGTGAAGTCGGAGTCGCTAGTAATCGCAGATCAG
CAACGCTGCGGTGAATACGTTCCCGGGCCTTGTACACACCGCCCGTCACG
TCATGAAAGTCGGTAACACCCGAAGCCGGTGGCCTAACCCCTTGTGGGAG
GCAGCCGTCGAAGGAATACCCCC

Following primer pairs and probe were designed:

| | Mb-16-603F: |
|---|---|
| (SEQ ID NO: 2) | |
| | GTAGGTGGTTTGTCGCGTTGTT |
| | Mb-16-834R: |
| (SEQ ID NO: 3) | |
| | CACCTAGTACCCACCGTTTACG |
| | Mb-16-897R: |
| (SEQ ID NO: 4) | |
| | TACTTAATGCGTTAGCTACGGCACG |
| | Mb-16-906R: |
| (SEQ ID NO: 5) | |
| | GGCGGGGTACTTAATGCGTTAGC |
| | Mb-16-1095R: |
| (SEQ ID NO: 6) | |
| | GCCATGCACCACCTGCAC |
| | Mb-16-1303R: |
| (SEQ ID NO: 7) | |
| | CCCTTTGTACCGGCCATTG |
| | Mb-16-700probe: |
| (SEQ ID NO: 8) | |
| | FAM-ATTCCTGGTGTAGCGG-MGB. |

### Example 2. Linear Range of Detection (Genomic DNA)

5 groups of detection systems were obtained through the design and screening of primer and probes to amplify eight Mycobacterium genomic DNAs, respectively, the concentration range of the standard curve was 100,000˜10 copies/reaction, and NTC was a sample-free negative quality control, i.e., DNA dilution, to verify the performance of the detection systems.

After comprehensively analyzing the amplification results of these five systems for different concentrations of templates, including the range of Ct values of the standard curve, R² and amplification efficiency of the standard curve, and the range of NTC Ct values, it was concluded that the 479 bp system (forward primer SEQ ID NO: 2, reverse primer SEQ ID NO: 6, and probe SEQ ID NO: 8) exhibited the best performance and was used in the subsequent experiments.

| | 252 | 298 | 305 | 479 | 676 |
|---|---|---|---|---|---|
| | bp | bp | bp | bp | bp |
| Mycobacterium neoformans | | | | | |
| 100000 copies / reaction | 18.3 | 18.4 | 18.5 | 19.1 | 19.8 |
| 10000 copies / reaction | 21.8 | 22.1 | 22.1 | 22.5 | 23.4 |
| 1000 copies / reaction | 25.3 | 25.4 | 25.3 | 26.1 | 27.1 |
| 100 copies / reaction | 29.0 | 28.7 | 28.9 | 29.3 | 30.1 |
| 10 copies / reaction | 32.0 | 32.0 | 31.6 | 33.0 | 35.5 |
| NTC | 33.1 | 33.3 | 38.3 | undet | undet |
| | 32.4 | 36.0 | 34.7 | undet | undet |
| | 36.3 | 34.5 | 43.9 | undet | undet |
| | 42.4 | 36.4 | 35.1 | undet | undet |
| Mycobacterium grassii | | | | | |
| 100000 copies / reaction | 18.7 | 19.2 | 19.1 | 19.3 | 21.5 |
| 10000 copies / reaction | 22.3 | 22.4 | 22.5 | 22.9 | 25.2 |
| 1000 copies / reaction | 25.6 | 25.9 | 25.9 | 26.4 | 28.5 |
| 100 copies / reaction | 29.1 | 29.1 | 28.9 | 29.7 | 32.4 |
| 10 copies / reaction | 32.2 | 33.0 | 32.1 | 33.7 | 35.0 |
| NTC | undet | 36.4 | undet | undet | undet |
| | undet | 34.4 | undet | undet | undet |
| | 35.6 | 35.3 | 41.8 | undet | undet |
| | undet | 34.4 | 34.7 | undet | undet |
| | undet | 34.9 | undet | undet | undet |
| | undet | 35.2 | 35.5 | undet | undet |
| Mycobacterium gordonii | | | | | |
| 100000 copies / reaction | 20.7 | 21.1 | 20.9 | 21.6 | 22.4 |
| 10000 copies / reaction | 24.1 | 24.4 | 24.0 | 25.0 | 25.8 |
| 1000 copies / reaction | 27.7 | 27.8 | 27.9 | 28.6 | 29.4 |
| 100 copies / reaction | 31.2 | 31.1 | 31.4 | 31.9 | 33.4 |
| 10 copies / reaction | 34.0 | 35.1 | 35.0 | 35.4 | 35.6 |
| NTC | undet | 35.6 | undet | undet | undet |
| | undet | undet | undet | undet | undet |
| | undet | undet | undet | undet | undet |
| | undet | 43.6 | undet | undet | undet |
| | undet | 36.1 | 43.4 | undet | undet |
| | 36.5 | 35.3 | undet | undet | undet |
| | 36.8 | 34.5 | 41.0 | undet | undet |
| | undet | 34.4 | undet | undet | undet |
| Mycobacterium kansasii | | | | | |
| 100000 copies / reaction | 21.0 | 21.2 | 21.2 | 22.0 | 22.8 |
| 10000 copies / reaction | 24.6 | 24.6 | 24.7 | 25.4 | 26.3 |
| 1000 copies / reaction | 27.7 | 27.7 | 28.1 | 28.9 | 30.1 |
| 100 copies / reaction | 31.5 | 30.4 | 31.4 | 32.2 | 33.5 |
| 10 copies / reaction | 33.3 | 31.0 | 34.7 | 34.2 | 36.6 |
| NTC | 37.0 | 31.4 | undet | undet | undet |
| | 34.7 | 30.7 | 36.2 | undet | undet |
| | 34.1 | 31.4 | undet | undet | undet |
| | 36.7 | 31.3 | 43.0 | undet | undet |
| | 36.1 | 31.4 | 38.1 | undet | undet |
| | 35.3 | 32.2 | 36.5 | undet | undet |
| Mycolicibacterium smegmatis | | | | | |
| 100000 copies / reaction | 20.1 | 20.2 | 20.0 | 20.6 | 21.4 |
| 10000 copies / reaction | 23.4 | 23.5 | 23.5 | 24.2 | 25.1 |
| 1000 copies / reaction | 27.1 | 26.8 | 26.8 | 27.5 | 28.5 |
| 100 copies / reaction | 30.1 | 29.4 | 30.2 | 31.0 | 32.0 |
| 10 copies / reaction | 34.5 | 30.8 | 34.2 | 34.0 | 35.7 |
| NTC | 34.6 | 31.0 | undet | undet | undet |
| | 34.6 | 31.2 | 40.7 | undet | undet |
| | 35.0 | 30.6 | 36.0 | undet | undet |
| | undet | 31.0 | undet | undet | undet |
| | undet | 31.7 | 35.4 | undet | undet |
| | 36.5 | 31.8 | undet | undet | 38.8 |
| Mycobacter foruitum | | | | | |
| 100000 copies / reaction | 19.3 | 19.4 | 19.5 | 20.3 | 21.3 |
| 10000 copies / reaction | 22.6 | 23.0 | 23.0 | 23.7 | 24.6 |
| 1000 copies / reaction | 26.3 | 26.4 | 26.3 | 27.1 | 29.4 |
| 100 copies / reaction | 29.3 | 29.6 | 29.3 | 30.1 | 31.6 |
| 10 copies / reaction | 32.1 | 32.5 | 32.9 | 33.0 | 35.0 |
| NTC | 33.0 | 34.3 | undet | undet | undet |
| | 33.8 | 34.2 | 36.8 | undet | undet |
| | 34.3 | 35.3 | 34.3 | undet | undet |
| | 33.4 | 33.3 | 34.2 | 36.7 | undet |
| | 34.7 | 33.2 | 34.5 | 34.3 | undet |
| | 33.1 | 35.1 | 34.5 | 33.7 | 39.9 |
| Mycobacterium scrofulaceum | | | | | |
| 100000 copies / reaction | 20.2 | 20.3 | 20.3 | 21.3 | 22.2 |
| 10000 copies / reaction | 23.7 | 23.9 | 23.9 | 24.6 | 25.7 |
| 1000 copies / reaction | 26.8 | 27.3 | 27.3 | 28.0 | 29.3 |
| 100 copies / reaction | 28.9 | 30.4 | 30.6 | 30.7 | 32.7 |
| 10 copies / reaction | 29.6 | 33.0 | 33.2 | 35.1 | 37.8 |
| NTC | 29.3 | 35.6 | undet | 37.1 | undet |
| | 29.4 | 33.5 | 34.7 | undet | 37.2 |
| | 29.4 | undet | 34.8 | 34.9 | undet |
| | 29.7 | 33.2 | 34.9 | undet | undet |
| | 29.4 | 33.1 | 34.7 | undet | 37.6 |
| | 29.4 | 33.1 | 36.2 | undet | undet |
| Mycobacterium intracellulare | | | | | |
| 100000 copies / reaction | 21.2 | 21.4 | 21.4 | 22.2 | 23.1 |
| 10000 copies / reaction | 24.6 | 24.9 | 24.8 | 25.6 | 26.4 |
| 1000 copies / reaction | 27.6 | 28.3 | 28.2 | 29.0 | 30.1 |
| 100 copies / reaction | 29.5 | 31.3 | 31.2 | 31.8 | 33.2 |
| 10 copies / reaction | 29.6 | 33.2 | 33..6 | 34.2 | 35.6 |
| NTC | 29.6 | 33.8 | 34.1 | 36.3 | 35.9 |
| | 29.5 | 33.3 | 35.6 | undet | undet |
| | 29.9 | 33.4 | 33.5 | undet | undet |
| | 29.8 | 33.2 | 35.4 | undet | undet |
| | 29.9 | 23.7 | 34.1 | undet | 38.1 |
| | 29.3 | 32.8 | 34.1 | 36.0 | undet |

### Example 3. Linear Range of Detection (Plasmid DNA)

In order to verify the performance of the 479 bp system (forward primer SEQ ID NO: 2, reverse primer SEQ ID NO: 6, probe SEQ ID NO: 8) again, a plasmid containing the target sequence was constructed as the standard, and diluted into following 7 concentration gradients using the DNA diluent gradient, and the NTC was a sample-free negative quality control, i.e., the DNA diluent.

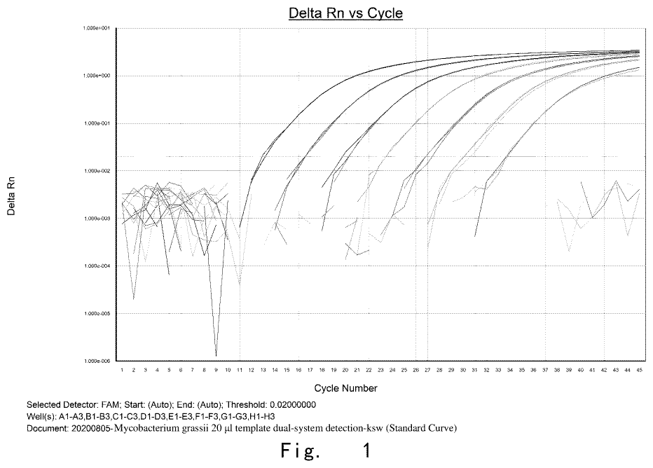

The results showed that the range of Ct values of the standard curve (3 replicate wells), R² of the standard curve and the amplification efficiency basically met the requirements when the concentration range of Mycobacterium grassii plasmid was 2×10⁷˜2×10¹ cp/reaction (shown in FIG. 1).

| Concentration of template | Ct value (in triplicate) | | |
|---|---|---|---|
| 2 × 10⁷copies / reaction | 12.9 | 12.9 | 13.1 |
| 2 × 10⁶copies / reaction | 16.5 | 16.5 | 16.5 |
| 2 × 10⁵copies / reaction | 20.2 | 19.7 | 20.2 |
| 2 × 10⁴copies / reaction | 23.3 | 23.4 | 23.4 |
| 2 × 10³copies / reaction | 27.3 | 26.8 | 26.8 |
| 2 × 10²copies / reaction | 30.1 | 30.1 | 30.5 |
| 2 × 10¹copies / reaction | 33.6 | 33.6 | 33.7 |

| | |
|---|---|
| NTC | undet |
| R² | 0.999 |
| Slope | −3.443 |
| Effect | 95.3% |

### Example 4. Test on Detection Limit

To verify the performance of the 479 bp system, the detection limit was tested. The plasmid containing the target sequence was diluted into three concentration gradients using the DNA diluent for testing.

The results showed that the detection rates of 20 cp/reaction, 10 cp/reaction, and 5 cp/reaction in 24 wells were 23/24, 24/24, and 16/24, respectively. The FAM Ct values ranged from 33.1 to undet, 33.3 to 36.6, and 33.5 to undet, respectively. The VIC Ct values ranged from 31.1 to 32.0, 31.0 to 32.0, and 30.9 to 32.3, respectively. The detection limit can basically reach 10 cp/reaction.

| | FAM | | | Detection rate |
|---|---|---|---|---|
| 20 cp/reaction | 36.4 | 33.9 | 33.9 | 23/24 |
| | 34.1 | 34.9 | 32.8 | |
| | 33.1 | 33.7 | 33.3 | |
| | 33.6 | 33.1 | 34.0 | |
| | 34.1 | 34.2 | 33.1 | |
| | 33.1 | 33.0 | undet | |
| | 34.4 | 33.2 | 33.9 | |
| | 33.2 | 33.8 | 33.5 | |
| | 34.5 | 34.8 | 34.4 | 24/24 |
| 10 cp/reaction | 34.5 | 33.3 | 33.3 | |
| | 35.3 | 33.5 | 34.7 | |
| | 34.0 | 36.3 | 33.2 | |
| | 34.5 | 33.5 | 35.0 | |
| | 36.6 | 33.9 | 33.3 | |
| | 35.2 | 34.7 | 33.5 | |
| | 33.7 | 35.1 | 34.7 | |
| | 33.5 | 44.3 | 34.1 | 16/24 |
| | 35.2 | 36.0 | 33.7 | |
| 5 cp/reaction | 36.3 | 34.8 | undet | |
| | undet | 35.2 | 35.3 | |
| | undet | 35.3 | 35.7 | |
| | 36.3 | undet | 34.3 | |
| | undet | 35.0 | 34.3 | |
| | undet | undet | 35.3 | |
| NTC | undet | undet | undet | / |

### Example 5. Test on Blank Limit

To verify the performance of the 479 bp system, the blank limit was tested. NTC was a negative quality control without samples, i.e. DNA diluent.

24 wells of NTC was tested, in which 22 wells were undet and 2 wells exhibited amplification, with a Ct value of 38.16 and 44.47, respectively. The negative rate was greater than 95%, which meets the requirements.

### Example 6. Test on Specificity

In order to verify the performance of the 479 bp system, genomic DNAs from a variety of common bacteria, fungi, Mycoplasma, engineered cells, and bacteria that have a close evolution relationship with Mycobacterium were selected and tested for specificity. The amount of the template was 30 ng/reaction. The tested species are listed below:

Types of sample

Common bacteria: Escherichia coli, Bacillus subtilis,
Staphylococcus aureus, Pseudomonas aeruginosa,
Clostridium spp.
Common fungi: Pichia pastoris, Candida albicans,
Aspergillus niger
Mycoplasma: Mycoplasma oralis, Mycoplasma
pneumoniae, Mycoplasma arginine, Mycoplasma
salivarius, Mycoplasma fermentans, Mycoplasma
porcineis, Mycoplasma fowleri, Mycoplasma
hominis, Mycoplasma leprae, Mycoplasma citricum
Engineered cells: CHO, Vero, 293T, NSO, MDCK
Bacteria with close evolutionary relationships:
Streptococcus pneumoniae, Streptomyces glacialis,
Amycolatopsis orientalis

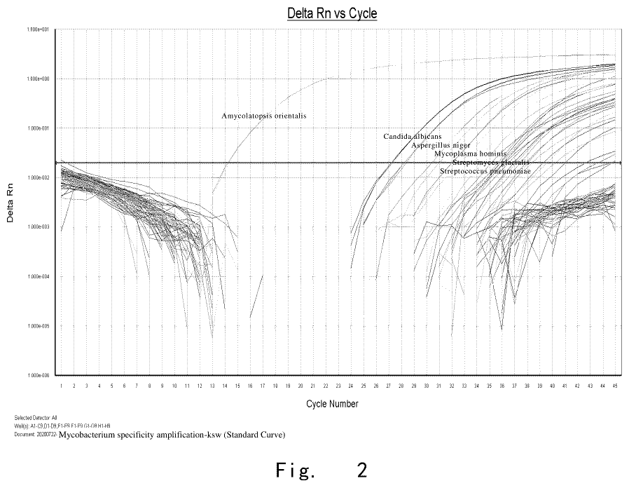

The results showed that Streptococcus pneumoniae, Streptomyces glacialis, Amycolatopsis orientalis, Candida albicans, Aspergillus niger, and Mycoplasma hominis exhibited amplification, with Ct values <35; other species exhibited Ct values ≥35 (see FIG. 2). It is concluded that, from the analysis of evolutionary relationships and the number of mismatched bases, Amycolatopsis orientalis orientalis may have a relatively strong interference since it belongs to Amycolatopsis, and belongs to Mycobacteriaceae as Mycobacteria, the sequences are more similar, and the number of mismatched bases is only 4.

| No. | Name | Ct value |
|---|---|---|
| 1 | Streptococcus pneumoniae | 34.8 |
| 2 | Streptomyces glacialis | 32.2 |
| 3 | Amycolatopsis orientalis | 14.1 |
| 4 | Candida albicans | 27.2 |
| 5 | Aspergillus niger | 28.2 |
| 6 | Mycoplasma hominis | 30.6 |

### Example 7. Extraction and Detection of 10 CFU Samples of Mycobacterium grassii Standard Strain without Cell Matrix

In order to verify the extraction performance of the extraction method on simple matrix samples, a 10 CFU sample of Mycobacterium grassii standard strain was tested. Steps for extracting nucleic acid of the sample are as follows:
1. 20 μl of proteinase K was added to the sample, shaken, mixed, and incubated at 55° C. for 60 minutes.
2. 200 μl of binding solution, 200 μl of isopropanol and 50 μl of magnetic beads were added, shaken for 5 minutes, and then placed on a magnetic separation rack.
3. After the solution was clarified and the magnetic beads were completely separated, the supernatant was carefully removed with a pipette tip.
4. 700 μl of washing solution A was added, shaken for 10 seconds, and placed on a magnetic separation rack.
5. After the solution was clarified and the magnetic beads were completely separated, the supernatant was carefully removed with a pipette tip, so as to complete the first magnetic bead washing.
6. 700 μl of washing solution B was added, shaken for 40 seconds, and placed on a magnetic separation rack.
7. After the solution was clarified and the magnetic beads were completely separated, the supernatant was removed with a pipette tip, so as to complete the second magnetic bead washing.
8. The tube cap was opened and dried at room temperature for 30 seconds to 3 minutes to remove any residual ethanol.
9. 50 μl of eluent was added along the wall of the centrifuge tube, shaken and mixed for 10 seconds.
10. The obtained mixture was incubated at 70° C. for 7 minutes, shaken and mixed again for 2-3 times during the incubation process.
11. After the incubation, the mixture was placed on a magnetic separation rack and after the magnetic beads were separated, the solution was carefully transferred to a clean centrifuge tube, so as to obtain the purified sample solution, which will be used as a template for the subsequent qPCR amplification.

The results showed that the detection rate of 24 samples was 24/24.

| | |
|---|---|
| Sample No. | Ct value |

| | | |
|---|---|---|
| 1 | 36.90 | 33.37 |
| 2 | 33.33 | 33.28 |
| 3 | 32.79 | 35.60 |
| 4 | 33.61 | 32.97 |
| 5 | 33.23 | 34.16 |
| 6 | 33.06 | 35.12 |
| 7 | 37.63 | 34.10 |
| 8 | 32.20 | 34.12 |
| 9 | 32.85 | 33.28 |
| 10 | 33.36 | 35.14 |
| 11 | 35.23 | 33.96 |
| 12 | 36.92 | 34.27 |
| 13 | 33.27 | 32.99 |
| 14 | 33.41 | 33.61 |
| 15 | 34.67 | 33.53 |
| 16 | 33.40 | 32.25 |
| 17 | 34.46 | 34.39 |
| 18 | 35.75 | 33.74 |
| 19 | 33.86 | 35.05 |
| 20 | 33.53 | 33.14 |
| 21 | 33.23 | 33.94 |
| 22 | 32.44 | 33.72 |
| 23 | 34.41 | 35.58 |
| 24 | 36.19 | 33.95 |

### Example 8. Detection of 10⁷ Vero Cells Matrix 100 CFU Mycobacterium grassii Standard Strain

In order to verify the extraction performance of the extraction method on complex matrix samples, a 10⁷ Vero cells matrix 10 CFU sample of Mycobacterium grassii standard strain was tested. Steps for extracting nucleic acid of the sample are as follows:
1. MB cell lysate (10:1 by volume) and a pretreatment solution (20:1 by volume) were added to the sample, vortexed, shaken and mixed.
2. After 5 mins, the obtained mixture was centrifuged at 450×g for 10 mins to precipitate cell debris, and the supernatant was aspirated and transferred into a new centrifuge tube.
3. 20 μl of proteinase K was added to the sample, shaken, mixed and incubated at 55° C. for 60 minutes.
4. 200 μl of binding solution, 200 μl of isopropanol and 50 μl of magnetic beads were added, shaken for 5 minutes, and then placed on a magnetic separation rack.
5. After the solution was clarified and the magnetic beads were completely separated, the supernatant was carefully removed with a pipette tip.
6. 700 μl of washing solution A was added, shaken for 10 seconds, and placed on a magnetic separation rack.
7. After the solution was clarified and the magnetic beads were completely separated, the supernatant was carefully removed with a pipette tip, so as to complete the first magnetic bead washing.
7. 700 μl of washing solution B was added, shaken for 40 seconds, and placed on a magnetic separation rack.
9. After the solution was clarified and the magnetic beads were completely separated, the supernatant was removed with a pipette tip, so as to complete the second magnetic bead washing.
10. The tube cap was opened and dried at room temperature for 30 seconds to 3 minutes to remove any residual ethanol.
11. 50 μl of eluent was added along the wall of the centrifuge tube, shaken and mixed for 10 seconds.
12. The obtained mixture was incubated at 70° C. for 7 minutes, shaken and mixed again for 2-3 times during the incubation process.
13. After the incubation, the mixture was placed on a magnetic separation rack and after the magnetic beads were separated, the solution was carefully transferred to a clean centrifuge tube, so as to obtain the purified sample solution, which will be used as a template for the subsequent qPCR amplification.

The results showed that the detection rate of 24 samples was 24/24.

| | |
|---|---|
| Sample No. | Ct value |

| | | |
|---|---|---|
| 1 | 32.32 | 33.36 |
| 2 | 32.63 | 33.34 |
| 3 | 32.61 | 33.49 |
| 4 | 32.44 | 32.99 |
| 5 | 33.12 | 33.86 |
| 6 | 34.02 | 33.77 |
| 7 | 32.50 | 33.88 |
| 8 | 33.08 | 33.83 |
| 9 | 33.66 | 33.82 |
| 10 | 33.16 | 33.83 |
| 11 | 33.23 | 33.38 |
| 12 | 33.73 | 33.40 |
| 13 | 36.84 | 34.43 |
| 14 | 35.05 | 33.88 |
| 15 | 32.49 | 32.67 |
| 16 | 32.68 | 33.34 |
| 17 | 33.23 | 33.85 |
| 18 | 33.44 | 33.51 |
| 19 | 33.41 | 34.31 |
| 20 | 34.44 | 34.12 |
| 21 | 33.41 | 34.30 |
| 22 | 34.34 | 33.94 |
| 23 | 33.31 | 33.24 |
| 24 | 34.87 | 33.82 |

### Example 9. Detection of 10⁷ 293T Cells Matrix 100 CFU Mycobacterium grassii Standard Strain

Example 8 was repeated, however, a 10⁷ 293T cells matrix 10 CFU sample of Mycobacterium grassii standard strain was used for testing.

The results were:

| | |
|---|---|
| Sample No. | Ct Value |

| | | |
|---|---|---|
| 1 | 32.44 | 32.40 |
| 2 | 33.11 | 32.63 |
| 3 | 31.55 | 32.49 |
| 4 | 32.64 | 32.75 |
| 5 | 33.14 | 32.94 |
| 6 | 33.74 | 33.16 |
| 7 | 33.18 | 33.82 |
| 8 | 32.90 | 35.35 |
| 9 | 31.94 | 32.55 |
| 10 | 33.44 | 33.08 |
| 11 | 32.66 | 32.39 |
| 12 | 31.83 | 33.65 |
| 13 | 31.22 | 31.37 |
| 14 | 32.08 | 32.14 |
| 15 | 33.12 | 33.62 |
| 16 | 33.88 | 37.36 |
| 17 | 32.26 | 32.54 |
| 18 | 32.03 | 33.30 |
| 19 | 32.86 | 33.76 |
| 20 | 33.98 | 32.74 |
| 21 | 32.66 | 33.51 |
| 22 | 32.35 | 32.95 |
| 23 | 34.47 | 33.06 |
| 24 | 33.85 | 33.41 |

### Example 10. Detection of 10⁷ Vero Cells Matrix 100 CFU Mycobacterium grassii Standard Strain

Example 8 was repeated, however, a 10⁷ Vero cells matrix 10 CFU sample of Mycobacterium grassii standard strain was used for testing.

The results were:

| | |
|---|---|
| Sample No. | Ct Value |

| | | |
|---|---|---|
| 1 | 33.55 | 33.93 |
| 2 | 33.96 | 34.11 |
| 3 | 35.29 | 36.64 |
| 4 | 35.61 | 34.17 |
| 5 | 33.62 | 34.36 |
| 6 | 34.43 | 34.24 |
| 7 | 34.56 | 35.41 |
| 8 | 35.55 | 35.15 |
| 9 | 38.15 | 37.71 |
| 10 | 37.69 | 39.25 |
| 11 | 37.37 | 36.47 |
| 12 | 36.93 | 35.81 |
| 13 | 44.35 | 38.23 |
| 14 | 35.68 | 36.17 |
| 15 | 36.50 | 36.70 |
| 16 | 36.63 | 36..49 |
| 17 | 37.43 | 37.47 |
| 18 | 38.69 | 34.17 |
| 19 | 33.54 | 35.35 |
| 20 | 36.31 | 34.81 |
| 21 | 35.81 | 34.75 |
| 22 | 34.65 | 34.82 |
| 23 | 36.29 | 37.16 |
| 24 | 38.33 | 36.02 |

### Example 11. Detection of 10⁷ 293T Cells Matrix 100 CFU Mycobacterium grassii Standard Strain

Example 8 was repeated, however, a 10⁷ 293T cells matrix 10 CFU sample of Mycobacterium grassii standard strain was used for testing.

The results were:

| | |
|---|---|
| Sample No. | Ct Value |

| | | |
|---|---|---|
| 1 | 40.37 | 34.03 |
| 2 | 35.43 | 36.17 |
| 3 | NoCt | 35.61 |
| 4 | NoCt | 36.22 |
| 5 | 37.54 | 34.43 |
| 6 | 36.30 | 36.16 |
| 7 | 36.06 | 35.05 |
| 8 | 35.26 | 35.04 |
| 9 | N/A | 39.66 |
| 10 | 40.17 | 37.16 |
| 11 | 34.92 | 37.55 |
| 12 | 36.26 | 35.48 |
| 13 | 35.65 | 35.85 |
| 14 | 33.97 | 35.11 |
| 15 | 36.28 | 36.05 |
| 16 | 36.58 | 37.12 |
| 17 | 35.15 | 35.10 |
| 18 | 34.52 | 35.18 |
| 19 | 35.12 | 35.99 |
| 20 | 34.72 | 35.03 |
| 21 | 36.10 | 35.50 |
| 22 | 36.41 | 36.19 |
| 23 | 36.57 | 35.26 |
| 24 | 41.47 | 34.81 |

All documents mentioned in herein are cited as references in the present application as if each document were cited individually as a reference. It is further to be understood that after reading the above teachings of the present invention, a skilled person may make various alterations or modifications to the present invention, and these equivalent forms will fall within the scope of the claims as appended to the present application.

1. A primer pair for detecting Mycobacterium genomic DNA, said primer pair comprising a forward primer and a reverse primer, wherein the forward primer binds to positions 510-550, preferably positions 518-539, of the sequence shown in SEQ ID NO: 1 on the Mycobacterium genomic DNA; the reverse primer binds to positions 740-780, 780-830, 790-830, 970-1010 or 1160-1200, preferably positions 748-769, 791-815, 800-822, 979-996, or 1175-1193, of the sequence shown in SEQ ID NO: 1 on the Mycobacterium genomic DNA; and the length of the product obtained through the amplification by said primer pairs is 240 to 700 bp, respectively.

2. The primer pair of claim 1, wherein, in the primer pair, the forward primer is shown in SEQ ID NO: 2 and the reverse primer is shown in any one of SEQ ID NO: 3-7.

3. The primer pair of claim 2, wherein, in the primer pair, the forward primer is shown in SEQ ID NO: 2 and the reverse primer is shown in SEQ ID NO: 6.

4. The primer pair of any one of claims 1-3, wherein the primer pair further comprises a probe, and the probe binds to positions 600-640, preferably positions 613-628 of the sequence shown in SEQ ID NO: 1 on the genomic DNA of Mycobacterium.

5. The primer pair of claim 4, wherein the probe is shown in SEQ ID NO: 8.

6. A detection reagent, comprising the primer pair of any one of claims 1-5.

7. A method for detecting genomic DNA of Mycobacterium, comprising: subjecting a sample to be tested to a PCR using the primer pair of any one of claims 1-5 or detection reagent of claim 6 and detecting a PCR amplification product.

8. A PCR kit, comprising a container and the primer pair of any one of claims 1-5 in the container.

9. A PCR method, comprising a step of:
amplifying a target product in a PCR detection system utilizing a primer pair of any one of claims 1-5.

10. Use of a primer pair of any one of claims 1-5 or an detection reagent of claim 6 of claim 6 for detecting the presence of Mycobacterium DNA in a subject to be tested.
